(19) United States
(12) Patent Application Publication
(10) Pub. No.: US 2012/0185031 A1
(43) Pub. Date: Jul. 19, 2012

(54) ROTARY AND LINEAR HANDLE MECHANISM FOR CONSTRAINED STENT DELIVERY SYSTEM

(76) Inventors: Michael Ryan, Limerick (IE); John Neilan, Galway (IE)

(21) Appl. No.: 13/346,195

(22) Filed: Jan. 9, 2012

Related U.S. Application Data
(60) Provisional application No. 61/434,245, filed on Jan. 19, 2011.

(51) Int. Cl. A61F 2/84

(52) U.S. Cl. 623/1.12

(57) ABSTRACT

A stent delivery system and a method for implanting a stent are provided. The system includes an elongate shaft including a proximal portion, a distal portion, a lumen extending at least partially therethrough, and a stent-receiving portion on the distal shaft portion. The system also includes a stent positioned at the stent-receiving portion of the elongate shaft, the stent having a constrained configuration and an expanded configuration. Proximal and distal constraining members releasably connected to the stent and having a first position and a second position are also included. The proximal and distal constraining members cooperatively apply longitudinal tensile force to at least a portion of the stent with the proximal and distal constraining members each in the first position. A drive system configured to simultaneously move the proximal and distal constraining members is provided, including a threaded drive shaft in mechanical communication with a rotatable handle.

### CROSS-REFERENCE TO RELATED APPLICATIONS

This application claims priority to both U.S. Provisional Patent Application No. 61/434,245, filed Jan. 19, 2011, which is incorporated herein by reference in its entirety.

### TECHNICAL FIELD

This invention relates to a medical device and, in particular to a device for delivering and deploying a stent and a method of delivering and deploying the stent into a body lumen.

### BACKGROUND

A self-expanding stent is typically introduced into a patient body using a delivery device that includes an outer sheath coaxially disposed and slidable over an inner catheter. The stent is disposed at the distal end of the device between the inner catheter and the outer sheath and held in a compressed position by the outer sheath. The inner catheter and the outer sheath move coaxially with respect to each other. The stent may be deployed by proximally pulling back the outer sheath relative to the inner catheter until the stent is exposed. The self-expanding stent expands from the stent distal end to the stent proximal end as the sheath is proximally withdrawn.

Several problems may occur with the sheathed delivery device described above. The sheath release delivery devices are difficult to reposition or remove and slow to operate. The stent may only be partially deployed prior to reconstrainment of the stent by the sheath in order to still reposition or remove the stent. After the stent is fully deployed (i.e., radially expanded), the sheath cannot reconstrain the stent. For example, utilizing a conventional outer sheath/inner catheter delivery device may cause the physician to inadvertently use excessive force and pull back the outer sheath too far, thereby prematurely deploying the stent in an incorrect position within a body lumen. At this step in the procedure, repositioning of the stent becomes difficult, if not impossible, because the stent has already radially self-expanded into the body lumen. Additionally, retraction of the outer sheath may not be achieved with controlled movement because the physician is manually retracting the outer sheath which may lead to uneven or inadvertent jerking back of the outer sheath that can lead to improper positioning of the stent.

Additionally, in a typical sheath release device where the outer sheath is proximally withdrawn, the first portion of the self-expanding stent to make contact with the body vessel is the most distal portion of the stent. This type of release may cause difficulty in accurately placing the proximal portion of the stent because the distal end of the stent is positioned first while the proximal portion of the stent is still covered by the outer sheath. Accurate placement of the proximal portion of the stent and/or the stent body may be important in certain applications, for example to prevent stent migration or to properly open a stricture along the entire length of the stricture. An additional drawback occurs with the sheathed stent delivery system where direct visualization of the stent is required. For example, in endoscopically placed stents, the sheath tends to prevent or obscure the location of the stent, making accurate placement of the stent more difficult.

Further potential drawbacks for the conventional sheathed stent delivery system involve the stent placement within the system prior to use within a patient. Loading and anchoring of a conventional sheathed stent delivery device is an involved process that may require preloading the stent into the device so that the stent remains compressed within the sheath during shipment and storage prior to use in the patient. Extended compression of the stent may lead to an alteration in the stent mechanical properties.

Conventional sheathed stent delivery devices also require a high force to overcome the friction between the stent and the sheath that may also be a problem for proper stent placement within the patient. The introducer must be mechanically stronger to overcome the frictional forces to avoid undesirable frictional consequences such as stretching of the introducer catchers and hysterics in the movement of the stent. The sheathed stent delivery device also requires more space within an endoscope compared to a sheathless device and also adds additional expense to the delivery system.

Accordingly, in view of the drawbacks of current technology, there is a desire for a delivery system that can increase the control, accuracy and ease of placement of a stent during deployment of the stent within a patient. A desirable delivery system will reduce the risk of malfunction while providing for a smooth, accurate, and quick deployment of the entire stent. The delivery system also will provide the ability to reconstrain, recapture, reposition, and/or remove the stent after expansion of the stent.

### SUMMARY OF THE INVENTION

In various aspects, embodiments of a stent delivery system may include an efficient mechanism for stent deployment and retraction. One embodiment of a stent delivery system may include an elongate outer tubular shaft including a proximal portion, a distal portion, and a stent attachment portion on the distal portion of the outer shaft, said stent attachment portion configured for attachment to a proximal stent end; an elongate inner shaft extending longitudinally, coaxially, slidably through the outer elongate tubular shaft including a proximal portion, a distal portion, and a stent receiving portion on the distal portion of the shaft, said stent receiving portion configured for attachment to a distal stent end; a housing configured to house a stent-deployment mechanism; and a stent deployment mechanism disposed in the housing, the mechanism including: a rotatable handle configured to be rotatable relative to the housing; a drive member having a first threaded portion and a second threaded portion, the drive member being disposed in mechanical communication with the rotatable handle and configured to be rotated about its longitudinal axis by said mechanical communication upon an actuation of the rotatable handle; a first drive-engaging member attached to a proximal portion of the inner shaft and comprising a first drive-engaging surface configured to engage the first threaded portion of the drive member; and a second drive-engaging member attached to a proximal portion of the outer shaft, and comprising a second drive-engaging surface configured to engage the second threaded portion of the drive member; and wherein the inner shaft and the outer shaft are configured to cooperatively apply opposing longitudinal tensile forces to at least a portion of a stent in the constrained configuration with the proximal and distal constraining members each in a first position corresponding to a first relative position of the movable handle, first drive-engaging member, and second drive-engaging member.

### BRIEF DESCRIPTION OF THE DRAWINGS

FIG. 1 is a side view of a stent delivery system according to one embodiment;

FIG. 2 is a longitudinal section view of the device shown in FIG. 1 showing the stent in a constrained configuration;

FIG. 3 is a longitudinal section view of the device shown in FIG. 2 with an outer sheath withdrawn and the stent in a constrained configuration;

FIG. 4 is a longitudinal section view of the device shown in FIG. 3 with the stent in an expanded configuration;

FIG. 5A is a partial side view of a proximal portion of the stent and the device shown in FIG. 4 illustrating a proximal constraining member;

FIG. 5B is a partial side view of a distal portion of the stent and the device shown in FIG. 4 illustrating a distal constraining member;

FIG. 5C is an enlarged view of a constraining member according to one embodiment;

FIG. 6A is a partial side view of an alternative embodiment of a proximal constraining member;

FIG. 6B is a partial side view of an alternative embodiment of a distal constraining member;

FIG. 6C is an enlarged view of an alternative embodiment of a constraining member;

FIG. 6D is a partial sectional view of a constraining member;

FIGS. 7A and 7B are longitudinal section views of a delivery system illustrating a stiffening member;

FIG. 8 is a partial longitudinal section view of a distal portion of a delivery system according to one embodiment;

FIGS. 9A-9D are cross sectional views of the delivery system shown in FIG. 8;

FIGS. 10A and 10B are longitudinal section views of a delivery system having alternative constraining members;

FIG. 11 is a side view of a stent deployment system;

FIG. 11A is a transverse section view of the system of FIG. 11 taken line 11A-11A; and

FIGS. 12A and 12B are longitudinal views (including partial longitudinal distal section views) of the system of FIG. 11, showing internal components and operation thereof.

### DETAILED DESCRIPTION OF THE EMBODIMENTS

The invention is described with reference to the drawings in which like elements are generally referred to by like numerals. The relationship and functioning of the various elements of this invention may better be understood by the following detailed description. However, the embodiments of this invention are not limited to the embodiments illustrated in the drawings. It should be understood that the drawings are not necessarily to scale, and in certain instances details have been omitted which are not necessary for an understanding of the present invention, such as conventional fabrication and assembly, and features described with reference to other embodiments.

As used in the specification, the terms proximal and distal should be understood as being from the perspective of a physician delivering the stent to a patient. Hence the term “distal” means the portion of the delivery system that is farthest from the physician and the term “proximal” means the portion of the delivery system that is nearest to the physician.

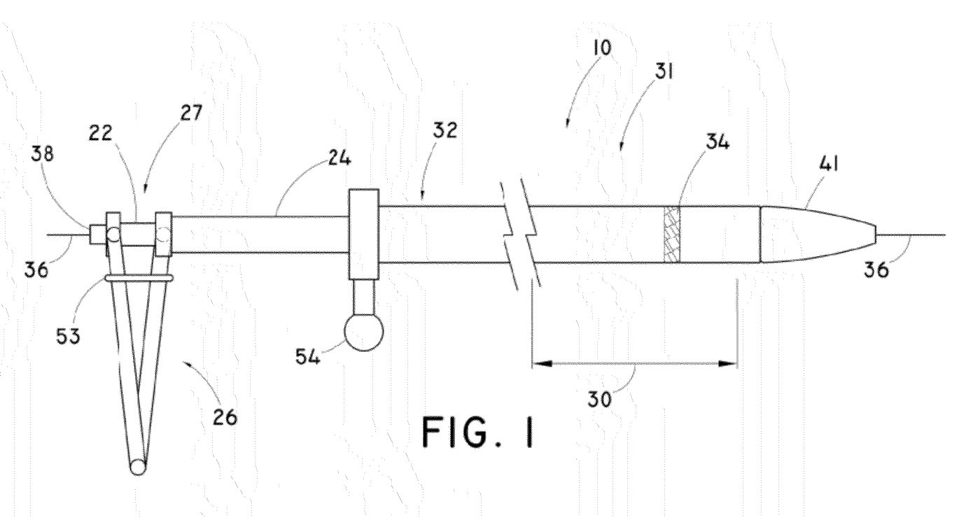

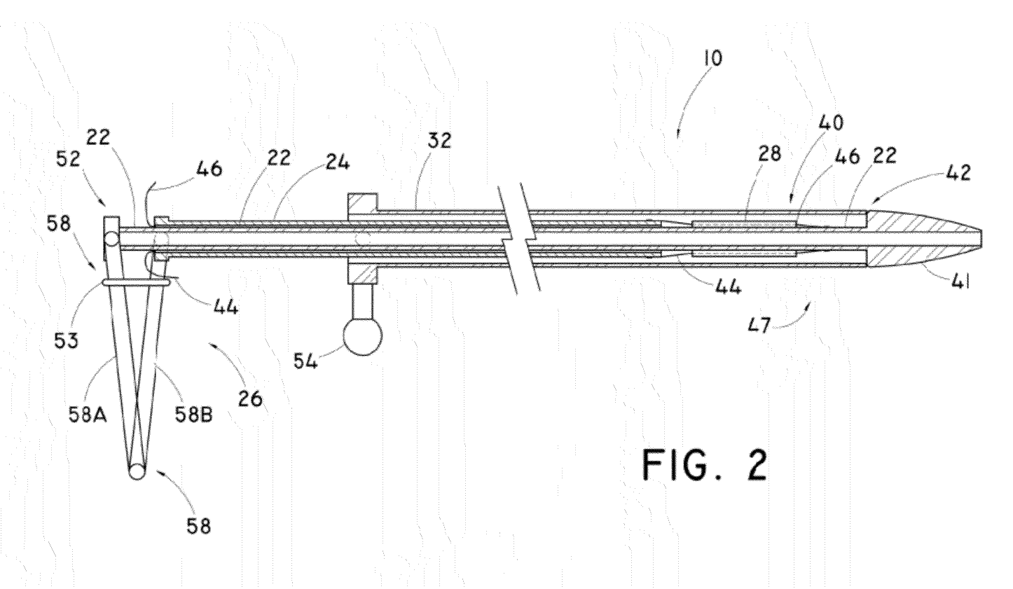

FIG. 1 illustrates a stent delivery system 10 for use in accordance with some non-limiting exemplary embodiments. The stent delivery system 10 includes an inner shaft 22, an outer shaft 24 and a handle 26 at a proximal portion 27 of the system 10. A stent 28 (shown in FIG. 2) is positionable on a stent region 30 of the inner shaft 22 at a distal portion 31 of the delivery system 10. The stent delivery system 10 may include an outer sheath 32 slidably positionable over a portion of the outer shaft 24 and the inner shaft 22 to cover the stent region 30 and the stent 28. One or more radio-opaque markers may be included on the delivery system 10 to indicate the position of the stent 28. The stent delivery system 10 may also include a stiffening member or wire guide 36 extendable through a port 38 of the inner shaft 22 through a distal tip 41 at the distal portion 31 of the delivery system 10.

FIG. 2 illustrates a sectional view of the stent delivery system 10 shown in FIG. 1. As shown in FIG. 2, the stent 28 is in a constrained configuration 40 collapsed against the inner shaft 22. In some embodiments, the stent 28 may be a self-expanding stent and may be configured—for example—as an esophageal stent. The stent 28 may be any kind of stent that has a tendency to radially collapse when a longitudinal force is applied to the ends of the stent proximally and distally outward along its central longitudinal axis (centerline). By way of non-limiting example, the stent 28 may be formed as a woven mesh formed from a metal or polymer or a laser cut pattern formed in a metal stent. The stent may also be formed from a bioabsorbable material. One example of a woven stent is the EVOLUTION® stent (Wilson-Cook Medical, Inc.)

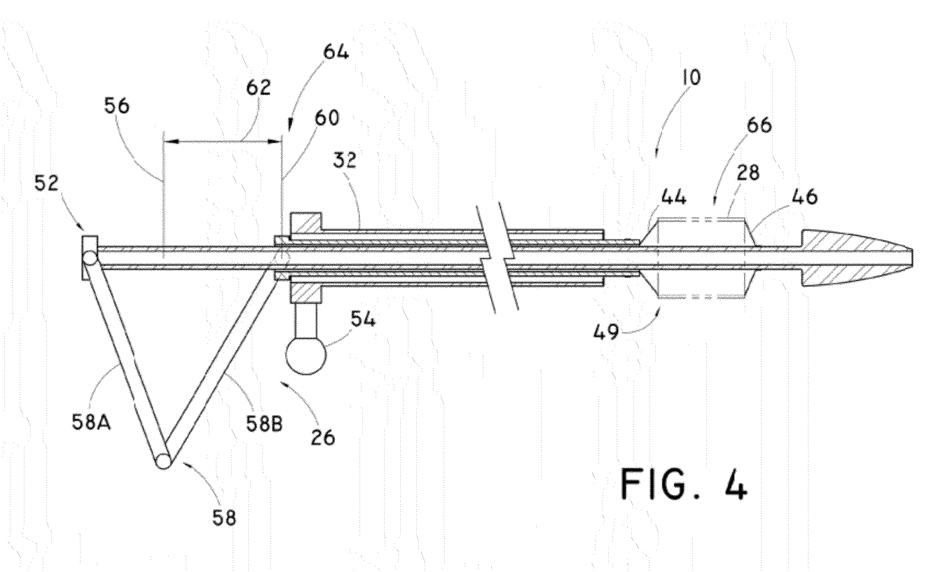

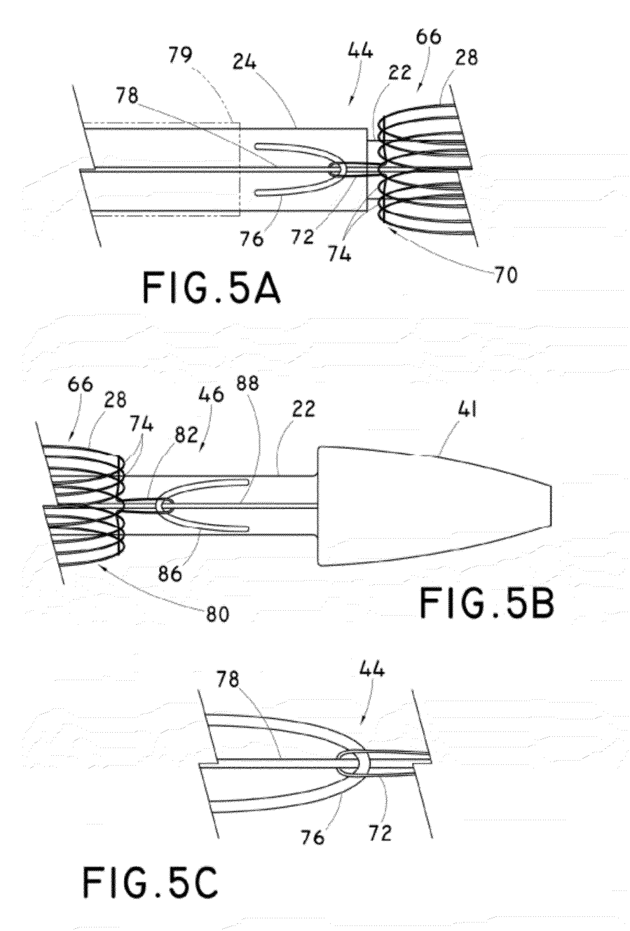

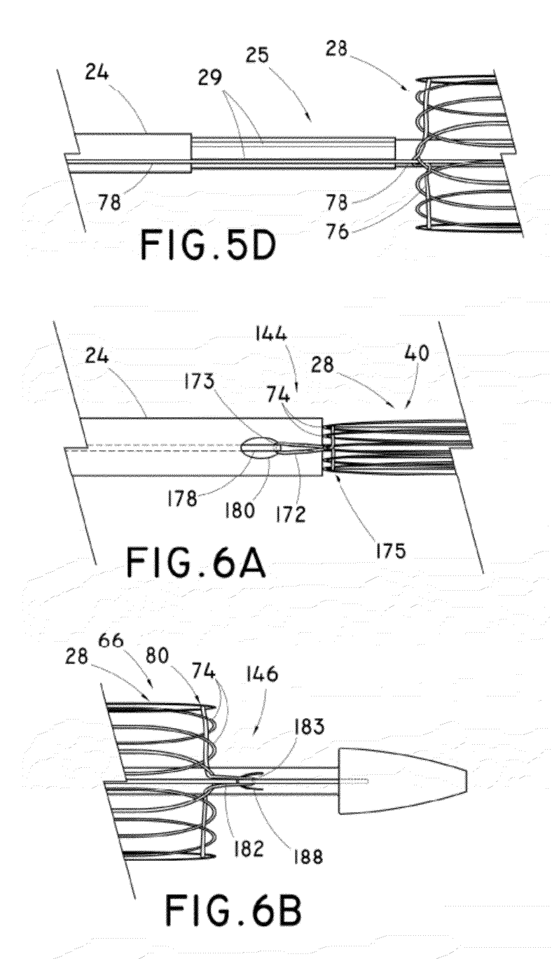

An outer sheath 32 that may be included is shown extended distally over the stent 28 and abutting the distal tip 41 of the inner shaft 22 forming a smooth outer surface 42 of the delivery system 10. The outer sheath 32 is operably connected to the handle 26. The outer sheath 32 may be provided to facilitate a smoother delivery of the system 10 through a body lumen of the patient. The stent 28 is held in the constrained configuration 40 by a different mechanism that may be provided with or without the outer sheath 32, one embodiment of which is described in detail below with reference to FIGS. 5A-5C. That embodiment includes a proximal stent constraining member 44 and a distal stent constraining member 46 configured to longitudinally constrain the stent 28 and hold the stent 28 collapsed against the inner shaft 22. The proximal and distal stent constraining members 44, 46 are operably connected to the handle 26 by connection of the proximal constraining member 44 to the outer catheter 24 and the distal constraining member 46 to the inner catheter 22. When present, the outer sheath 32 may provide some compressive force to the stent in addition to the proximal and distal constraining members 44, 46. The handle 26 is shown FIG. 2 in a closed position 52. The handle 26 may include a lock 53 to releasably lock the handle 26 in the closed position 52.

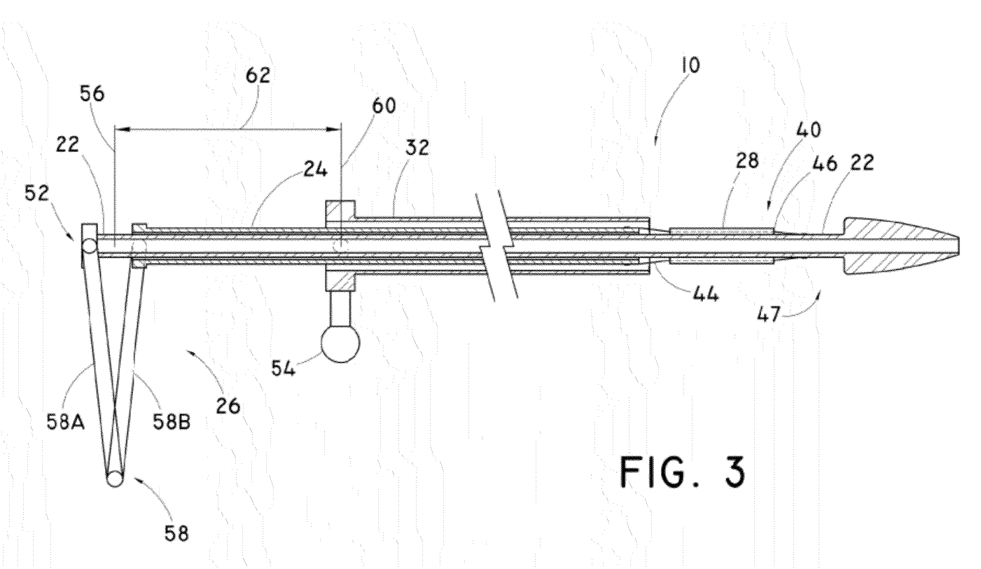

As shown in FIG. 3, the outer sheath 32 has been proximally pulled back, completely exposing the stent 28 in the constrained configuration 40 on the inner shaft 22. The outer sheath 32 may be releasably locked against the handle 26 to keep the sheath 32 stationary relative to the handle 26. The stent 28 is held compressed against the inner shaft 22 by the proximal and distal stent constraining members 44, 46 in a first position 47 applying longitudinal force to the stent 28 in opposite directions (which may be described as “central constraint” as the function is not accomplished by any external encompassing structure). The handle 26 is in the closed position 52 and the outer sheath 32 has manually been pulled proximally away from the stent region 30 of the inner shaft 22 and anchored to the sheath controlling portion 54 of the handle 26 to expose the stent 28.

The handle 26 further includes a proximal handle portion 58 that is operably connected to the inner shaft 22 and the outer shaft 24 to move the inner and outer shafts 22, 24 relative to each other as discussed below. The proximal handle portion 58 is movable between the closed position 52 (shown in FIG. 3) and an open position 64 (shown in FIG. 4). A midpoint 56 for attachment of the proximal handle portion 58 is shown for the handle 26. A second attachment midpoint 60 is shown for the sheath controlling portion 54. A distance 62 between the attachment midpoints 56, 60 remains constant when the proximal handle portion 58 is moved from the closed position 52 shown in FIG. 3 to the open position 64 shown in FIG. 4.

The stent 28 is shown in an expanded configuration 66 in FIG. 4 where the stent 28 is expanded away from the inner shaft 22. The proximal and distal constraining members 44, 46 are in a second position 49 and remain connected to the stent 28 but the longitudinal force on the stent 28 has been removed to allow the stent 28 to expand. The proximal portion 58 of the handle 26 has been moved to the open position 64 by expanding arms 58a, 58b of the proximal handle portion 58 in equal and opposite directions. The inner shaft 22 and the outer shaft 24 are moved in equal and opposite directions relative to each other by the proximal handle portion 58 and the proximal and distal constraining members 44, 46 are moved closer together. The stent 28 is released from the constrained configuration 40 to the expanded configuration 66 in response to the equal and opposite motion of the opening of the proximal handle portion 58 so that the release of the tension on the stent 28 is uniform within the patient lumen.

The proximal handle portion 58 may be spring loaded to facilitate the expansion of the arms 58a, 58b to the open position 64. The proximal handle portion 58 moves the inner shaft 22 relative to the outer shaft 24 so that the longitudinal tension exerted on the stent 28 by the proximal and distal constraining members 44, 46 is relaxed when the members 44, 46 are closer together and the stent 28 expands uniformly due to the uniform release of the tension on the stent 28 by the proximal and distal constraining members 44, 46.

As shown in FIG. 4, the proximal and distal constraining members 44, 46 remain connected to the stent 28 in the expanded configuration 66. The connection allows the stent 28 to be moved from the expanded configuration 66 with the outer sheath 32 completely removed from the stent 28 to the constrained configuration 40 so that the stent 28 is recollapsed onto the inner shaft 22 by moving the proximal handle portion 58 to the closed position 52. The proximal handle portion 58 moves the inner shaft 22 and the outer shaft 24 relative to each other so that the proximal and distal constraining members 44, 46 are spaced further apart and the longitudinal tension is returned to the stent 28 to collapse the stent onto the inner shaft 22. The stent 28 may be repeatedly moved between the constrained configuration 40 and the expanded configuration 66 by moving the proximal handle portion 58 between the closed position 52 and the open position 64 until the stent is properly positioned. With the stent repositioned in the constrained configuration 40, the outer sheath 32 may be repositioned over the stent 28 as shown in FIG. 2 and the stent 28 may even be withdrawn from the patient, for example if an incorrect size of stent was originally selected. The stent configurations may be changed multiple times within the patient for repositioning or removal until the proximal and distal constraining members 44, 46 are released from connection with the stent 28 as described below.

FIGS. 5A-5C illustrate an exemplary embodiment of a proximal constraining member 44 (FIG. 5A) and a distal constraining member (FIG. 5B). An exploded view of the components of the proximal constraining member 44 is shown in FIG. 5C and the components of the distal constraining member 46 may be a mirror image of the components of the proximal constraining member 44 (not shown). As shown in FIG. 5A, a proximal end portion 70 of the stent 28 remains connected to the inner shaft 22 even in the expanded configuration 66 using the proximal constraining member 44 in combination with the distal constraining member 46. The proximal constraining member 44 may include a first loop 72 that may be interwoven through one or more peaks 74 of the stent 28 so that the first loop 72 when pulled taught will collapse the peaks 74 of the stent 28 onto the inner shaft 22. The proximal constraining member 44 may further include a second retaining loop 76 that may be attached to the outer shaft 24.

The proximal constraining member 44 may also include a proximal retaining wire 78 that is configured to cooperate with the first loop 72 and the second retaining loop 76 to releasably lock the first loop 72 to the second retaining loop 76 to allow selective expansion and contraction of the stent 28 when the proximal handle portion 58 is moved between the open position 64 and the closed position 52 in cooperation with the distal constraining member 46. The first loop 72, the second loop 76 or both may be anchored at one or more points to better secure the stent 28 on the inner catheter 22, for example in a system 10 that is provided without a sheath. In some embodiments, the first loop 72 may be wound around the inner catheter 22 or the outer shaft 24 to facilitate holding the stent to the inner catheter 22 as the delivery system 10 is advanced to the treatment site through a curve, for example through an elevator of a duodenal endoscope.

An exemplary cooperative configuration of the proximal constraining member 44 is shown in FIG. 5C where a portion of the first loop 72 and the second retaining loop 76 are overlapping and the proximal retaining wire 78 extends through the overlapping loops 72, 76 to releasably hold the two loops 72, 76 together. The proximal retaining wire 78 shown in FIG. 5A may be frictionally engaged with a portion of the outer shaft 24 to hold the proximal retaining wire 78 in position until the stent 28 is in the proper position for release as discussed above. The proximal retaining wire 78 may be proximally withdrawn to release the proximal constraining member 44 and to completely release the stent 28 from connection to the inner shaft 22.

As shown in FIG. 5B, a distal end portion 80 of the stent 28 may remain connected the inner shaft 22 even in the expanded configuration 66 using the distal constraining member 46. The distal constraining member 46 may include a first loop 82 that may be interwoven through one or more peaks 74 of the stent 28 so that the first loop 82 when pulled taught will collapse the peaks 74 of the stent 28 onto the outer shaft 24. The distal constraining member 46 may further include a second retaining loop 86 that may be attached to the inner shaft 22. The first loop 82, the second loop 86 or both may be anchored at one or more points to better secure the stent 28 on the inner catheter 22, for example in a system 10 that is provided without a sheath. In some embodiments, the first loop 82 may be wound around the inner catheter 22 or the outer shaft 24 to facilitate holding the stent to the inner catheter 22 as the delivery system 10 is advanced to the treatment site through a curve similar to the loop 72 described above.

The distal constraining member 46 may also include a distal retaining wire 88 that is configured to cooperate with the first loop 82 and the second retaining loop 86 to releasably hold the loops 82, 86 together to allow selective expansion and contraction of the stent 28 when the proximal handle portion 58 is moved between the open position 64 and the closed position 52. The distal retaining wire 88 may be frictionally engaged with the inner shaft 22 or the distal tip 41 to hold the distal retaining wire 88 in position until the stent 28 is properly positioned for release. The distal constraining member 46 may be configured similarly to the proximal constraining member 44 shown in FIG. 5C with the distal retaining wire 88 releasably locking the first loop 82 and the second retaining loop 86 together. The distal retaining wire 88 may be proximally withdrawn to release the distal constraining member 46 and to completely release the stent 28 from connection to the inner shaft 22.

The proximal and distal retaining wires 78, 88 may be connected to the handle 26 for proximal withdrawal from the loops 72, 76, 82, 86. The withdrawal of the proximal and distal retaining wires 78, 88 may be simultaneous or sequential. Because the stent 28 has been positioned in the proper position within the lumen of the patient by equal and opposite movement of the handle 26 to the open position 64 allowing the stent 28 to move to the expanded configuration 66, the timing of the release of the retaining wires 78, 88 is not critical for the positioning of the stent 28. As will be understood by one skilled in the art, the proximal constraining member 44 may be connected to the inner catheter 22 and the distal constraining member 46 may be connected to the outer catheter 24. In embodiments provided without the outer sheath 32, the peaks 74 of the stent 28 are collapsed closely against the inner catheter 22 at both ends of the stent 28 for delivery to the patient site.

While the proximal and distal restraining members 44, 46 have been described with reference to connection to the proximal and distal end portions 70, 80 of the stent 28, it is also possible to provide proximal and distal constraining members 44, 46 that are connected to other portions of the stent 28 and still provide a constrained configuration 40 for the stent 28. For example, the proximal constraining member may be connected to a mid proximal portion or mid-point of the stent and the distal constraining member may be connected to the distal end portion of the stent. Similarly, the proximal constraining member may be connected to the proximal end portion of the stent and the distal constraining member may be connected to the midpoint of mid distal portion of the stent or both the proximal and distal constraining members may be connected to other than the proximal and distal end portions of the stent. In some embodiments, the proximal or the distal constraining members or both proximal and distal constraining members may be connected to the stent at a plurality of positions on the stent.

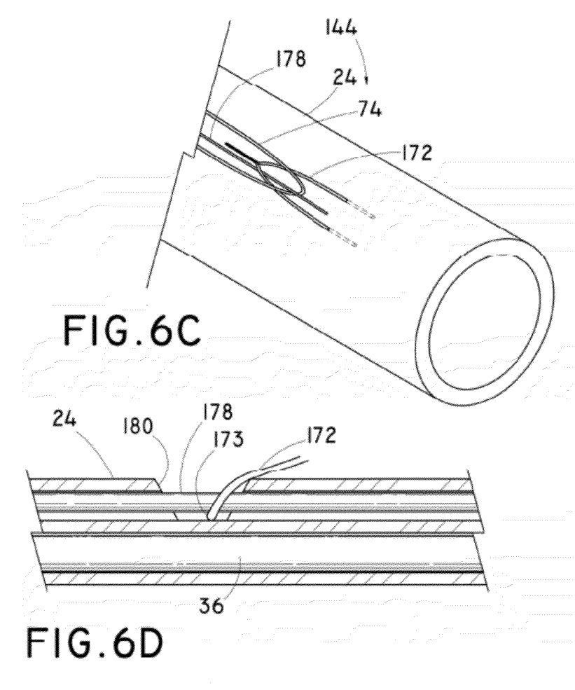

In some embodiments, the stent delivery system 10 may be provided with a proximal constraining member 144 and a distal constraining member 146 as shown in FIGS. 6A and 6B. Similar to the proximal and distal constraining members 44, 46 described above the proximal and distal constringing members 144, 146 cooperatively apply and release tensioning force on the stent 28 in connection with the handle 26. The proximal constraining member 144 is shown in FIG. 6A with stent 28 in the constrained configuration 40. The proximal constraining member 144 includes a first loop 172 and a proximal retaining wire 178. The first loop 172 may be connected to the outer catheter 24. By way of non-limiting example, one portion 173 of the first loop 172 may be connected to the outer catheter 24 through an opening 180 in the outer catheter 24 so that the portion 173 of the loop 172 is constrained under the proximal retaining wire 178 as shown in FIG. 6C. The first loop 172 may also be connected to the outer catheter 24 by welding, gluing, bonding or other fastening method known to one skilled in the art. Another portion 175 of the first loop 172 may be woven through one or more peaks 74 of the stent 28 so that the first loop 172 when pulled taught will collapse the peaks 74 of the stent 28 onto the inner shaft 22 as described above. The proximal constraining member 144 may also include a proximal retaining wire 178 that cooperatively engages a portion of the first loop 172 to releasably hold the first loop 172 on the stent 28 to allow the stent 28 to be expanded and collapsed repeatedly for proper positioning within the patient lumen. The proximal retaining wire 178 may be proximally withdrawn from the first loop 172 to release the stent 28 from connection with the proximal constraining member 144. The first loop 172 may be withdrawn with the device 10 from the patient and released from the stent 28.

The distal constraining member 146 is shown in FIG. 6B with stent 28 in the expanded configuration 66. The distal constraining member 146 includes a first loop 182 and a distal retaining wire 188. A portion 183 of the first loop 182 may be connected to the inner catheter 22 in a similar manner to the first loop 172 of the proximal constraining member 144 described above. Another portion 185 of the first loop 182 may be woven through one or more peaks 74 at the distal end 80 of the stent 28 so that when the first loop 182 of the distal constraining member 146 is pulled taught will collapse the peaks 74 of the stent 28 onto the inner shaft 22 as described above. The distal constraining member 146 may also include the distal retaining wire 188 that cooperatively engages a portion of the first loop 182 to releasably hold the first loop 182 on the stent 28 to allow the stent 28 to be expanded and collapsed repeatedly in cooperation with the proximal constraining member 144 for proper positioning within the patient lumen. The distal retaining wire 188 may be proximally withdrawn from the first loop 182 to release the stent 28 from connection with the distal constraining member 146. The first loop 182 may be withdrawn with the device 10 from the patient and released from the stent 28. As will be understood by one skilled in the art, the proximal constraining member 144 may be connected to the inner shaft 22 and the distal constraining member 146 may be connected to the outer shaft 24 and be movable in equal and opposite directions by operation of the proximal portion 58 of the handle 26.

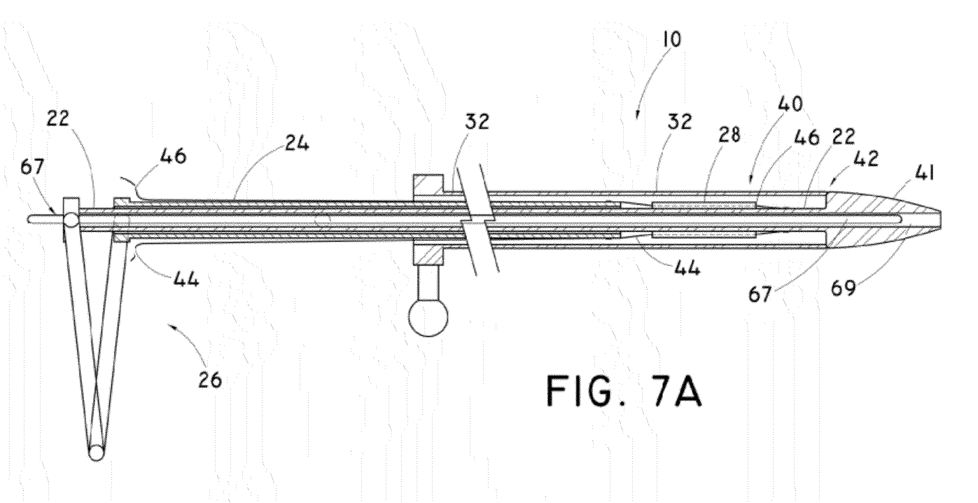

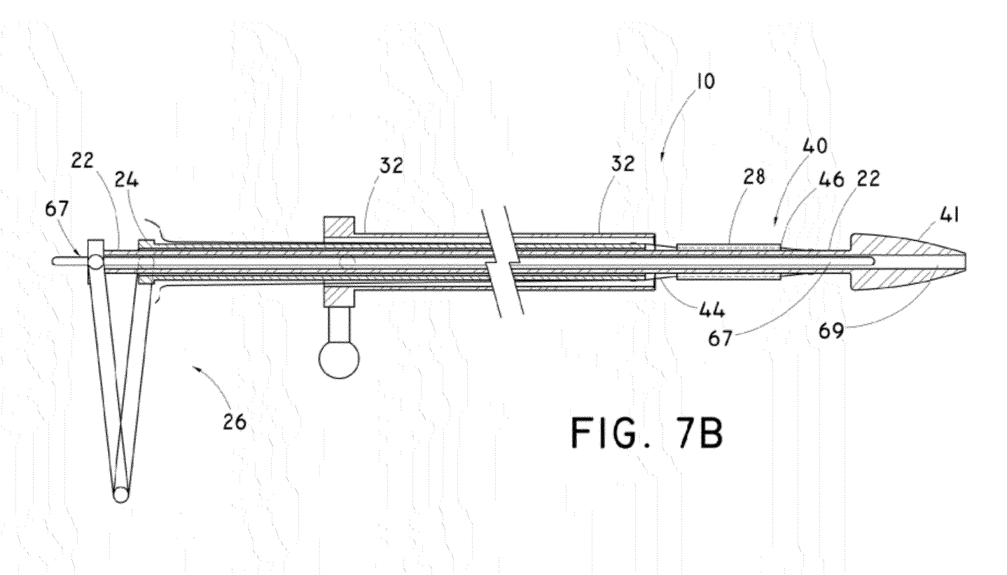

In some embodiments, a stiffening member 67 may be removably provided in a lumen 69 of the inner shaft 22 as shown in FIGS. 7A and 7B. The stiffening member may be provided as a mandrel, catheter, rod and the like that is removably insertable into the lumen 69. The stiffening member 67 may be provided to help increase the rigidity of the inner catheters 22 against the inward tensioning force of the stent 28 when the stent 28 is in the constrained configuration 40. In some embodiments, the inner shaft 22 may be provided in a soft material to facilitate passage through the body lumen. In the event that the materials are sufficiently soft, the inner catheter 22 may collapse or deform in response to the tensioning force of the stent 28 provided by the first and second constraining members 44, 46 longitudinally constraining the stent 28 against the inner shaft 22. The stiffening member 67 may be made from any material having suitable stiffness to provide support for the inner shaft 22 with the stent 28 longitudinally tensioned on the inner shaft 22. Exemplary materials for forming the shaft include, but are not limited to, metal alloys such as stainless steel, tantalum or its alloys, tungsten, platinum, gold, copper, palladium, rhodium, or a superelastic alloys, such as nitinol or polymers that can be provided with sufficient shore hardness, such as Pebax, Peek, polyimide, liquid crystal polymers (LCP) such as Vectran, polyethylene, polyethylene terephthalate and Nylon.

As shown in FIG. 7A, the outer sheath 32 may be provided for delivery of the stent to the area of the treatment site. The outer sheath 32 compresses the stent against the inner shaft 22 for delivery of the device 10 to the treatment site with the stiffening member 67 removed and the stent 28 in the constrained configuration 40. (See FIG. 1.) The stiffening member 67 may be inserted into the lumen 69 when the stent 28 is near the proper position for implantation into the patient and the outer sheath is over the stent 28 as shown in FIG. 7A. The outer sheath 32 may be withdrawn and the stent 28 remains constrained on the inner shaft 22 by the proximal and distal constraining members 44, 46. The stiffening member 67 supports the inner shaft 22 against the compressive tensioning force exerted by the proximal and distal constraining members 44, 46.

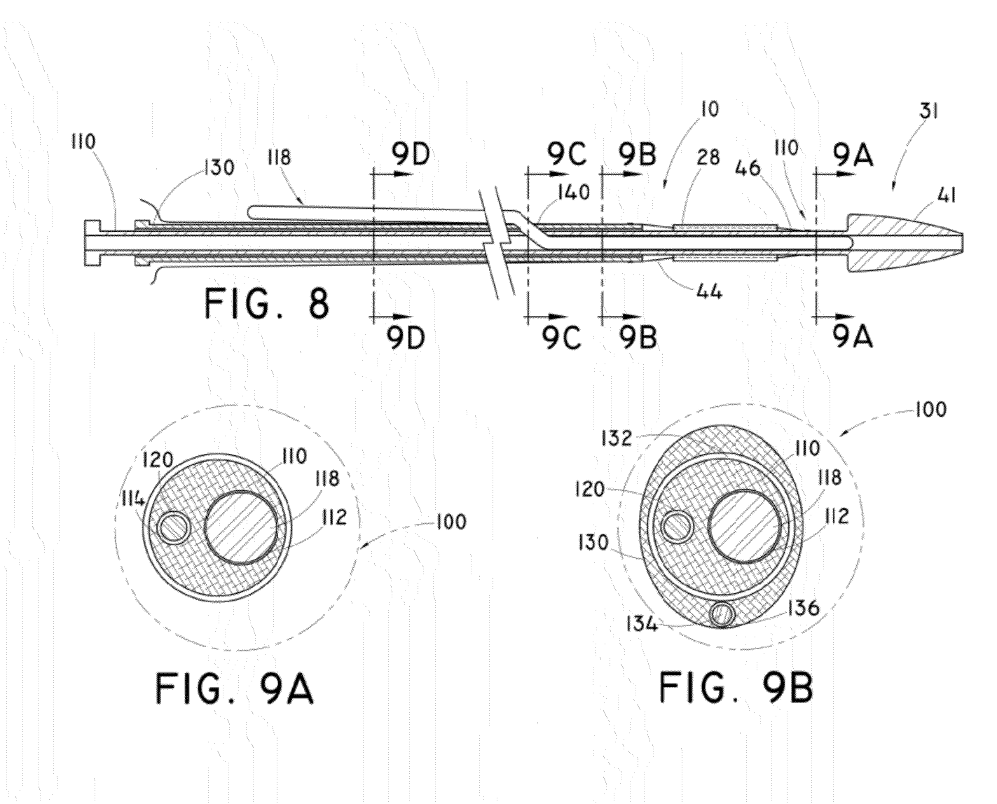

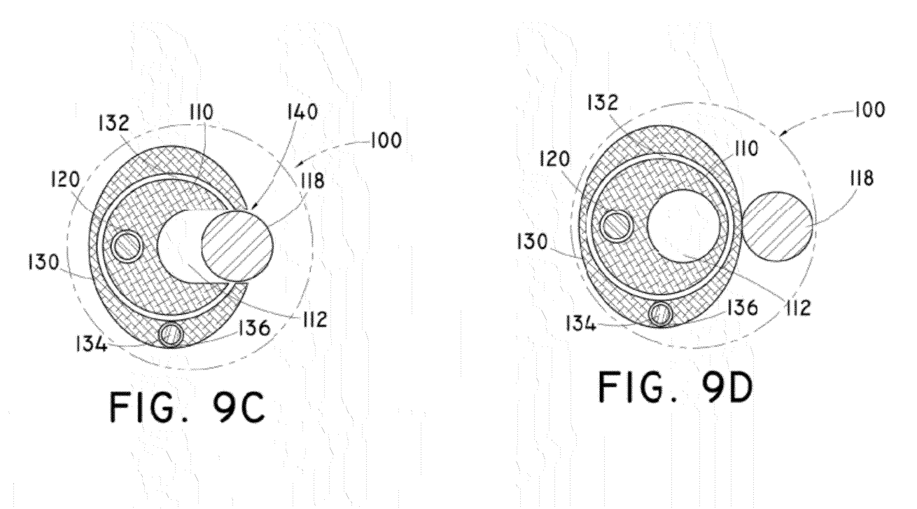

FIG. 8 illustrates a sectional view of the distal portion 31 of the stent delivery device 10 provided in a rapid exchange configuration. FIGS. 9A-9D show cross sectional views of an exemplary lumen configuration through the device 10 along different portions indicated in FIG. 8 in relation to a working channel of an endoscope. Many other lumen configurations are possible with the stent delivery device 10 and the following discussion is provided by way of non-limiting example. A working channel 100 of an endoscope is represented by the dashed line in FIGS. 9A-9D. FIG. 9A shows the cross sectional view along line A-A of FIG. 8 that is distal to the stent 28. The cross section view in FIG. 9A illustrates an inner catheter 110 having a first lumen 112 and a second lumen 114. A guide wire 118 is shown in the first lumen 112 and a first retaining wire 120 is shown in the second lumen 114. The first retaining wire 120 is a component of the distal constraining member 46. FIG. 9B shows the cross sectional view along line B-B of FIG. 8 taken proximal to the stent 28 and shows the inner shaft 110 within a first lumen 132 of an outer shaft 130 in relation to the working channel 100. A second retaining wire 134 is shown within a second lumen 136 of the outer catheter 130. The second retaining wire 134 is a component of the proximal constraining member 44 shown in FIG. 8.

FIG. 9C illustrates the cross sectional view taken along line C-C of FIG. 8. FIG. 9C illustrates a rapid exchange port 140 within a distal portion 31 of the device 10. The rapid exchange port 140 provides access to the first lumen 132 of the outer shaft 130 and to the first lumen 112 of the inner shaft 110. As shown in FIG. 9C, the guide wire 118 is being exchanged in the rapid exchange port 140. Any other type of device suitable for insertion into a rapid exchange port may also be inserted into the rapid exchange port 140. By way of non-limiting example, the stiffening member 67 described above may be inserted into the rapid exchange port 140 to provide additional stiffness to support the stent 28 on the inner shaft 22 in the longitudinally tensioned constrained configuration 40 as discussed above with reference to FIG. 7B.

FIG. 9D illustrates the cross sectional view taken along line D-D in FIG. 8 proximal to the rapid exchange port 140. FIG. 8D illustrates the wire guide 118, or other device suitable for insertion into the rapid exchange port 140, external to the outer shaft 130 and within the working channel 100 of the endoscope. The inner shaft 110 is enclosed within the first lumen 132 of the outer catheter 130.

The stent delivery system 10 may also be provided in an over-the wire configuration, for example, as shown in FIG. 1. In the over-the-wire configuration, the first lumen 112 of the inner shaft 110 is accessible from the proximal end portion of the inner shaft 110. In the over-the-wire configuration, the cross sectional views taken along the lines C-C and D-D would be the same as the cross-sectional view taken along line B-B as shown in FIG. 9B.

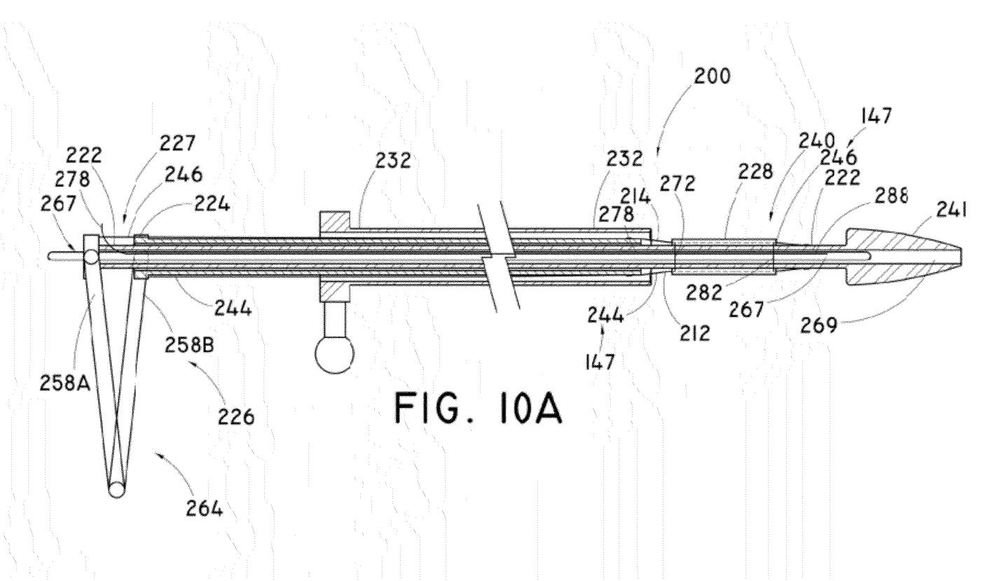

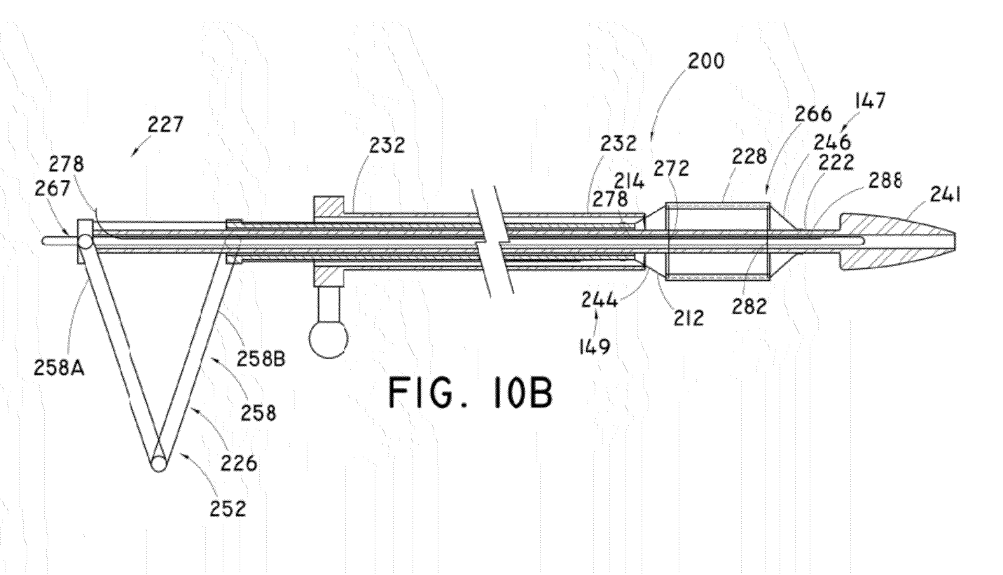

As shown in FIGS. 10A and 10B, a stent delivery system 200 may be provided with two wires 212, 214 to control the expansion and contraction of a stent 228. The stent delivery system 200 includes an inner shaft 222 and a handle 226 at a proximal portion 227 of the system 200. The stent 228 is positionable on the inner shaft 222 at a distal portion 231 of the stent delivery system 200. The stent delivery system 200 may include an outer sheath 232 slidably positionable over a portion of the inner shaft 222 to cover the stent 228. The stent delivery system 200 may also include a stiffening member 267 similar to the stiffening member 67 described above with reference to FIGS. 7A and 7B. The stent 228 is shown in FIG. 10A in a constrained configuration 240. Similar to the stent 28 described above, the stent 228 is movable between the constrained configuration 240 and an expanded configuration 266 shown in FIG. 10B. The stent 228 is moved between the constrained and expanded configuration with a proximal constraining member 244 and a distal constraining member 246.

The proximal and distal constraining members 244, 246 cooperatively apply and release longitudinal tension on the stent 228 to move the stent between the constrained configuration 240 and the expanded configuration 266. In the embodiment shown in FIGS. 10A and 10B, the wires 212 and 214 of the proximal and distal constraining members 244, 246, respectively move in equal and opposite directions in connection with arms 258a, 258b of the handle portion 258 moving in equal and opposite directions. By way of non-limiting example, the handle 226 is shown in an open position 264 that holds the proximal and distal constraining members 244, 246 apart in a first position 147 to apply longitudinal force to the stent 228 to hold the stent 228 against the inner shaft 222 in the constrained configuration 240 as shown in FIG. 10A. In FIG. 10B, the arms 258a, 258b are moved to a closed position 252 and the proximal and distal constraining members 244, 246 are moved closer together in a second position 149 and release the tension on the stent 228 so the stent 228 moves to an expanded configuration 226 with the proximal and distal constraining members 244, 246 still connected to the stent 228. Similar to the embodiments described above, the stent 228 may be moved between the expanded and constrained configurations 266, 240 multiple times until the correct position within the patent's lumen is obtained.

The proximal constraining member 244 may include the wire 212, a loop 272 and a proximal retaining wire 278. The wire 212 may be provided with a loop to overlap with the loop 272 so that the proximal retaining wire 278 may releasably lock the wire 212 and the loop 272 together until the proximal retaining wire 278 is withdrawn. The distal constraining member 246 may be provided with the wire 214, a loop 282 and a distal retaining wire 288 in a similar arrangement to the proximal constraining member 244. The proximal and distal retaining wires 278, 288 may be proximally withdrawn to completely release the stent 228 when the stent 228 is properly positioned.

The materials used to manufacture the components of the stent delivery systems described herein may be any materials known to one skilled in the art that are suitable for use in patients. By way of non-limiting example, the shafts and sheaths may be formed from polytetrafluoroethylene (PTFE) particularly when a low friction outer sheath is desirable. Nylon and HDPE may also be used for clarity. Additional possible materials include, but are not limited to the following, polyethylene ether ketone (PEEK), fluorinated ethylene propylene (FEP), perfluoroalkoxy polymer resin (PFA), polyamide, polyurethane, high density or low density polyethylene, and nylon including multi-layer or single layer structures and the like and may also include reinforcement wires, braid wires, coils, coil springs and or filaments. The stent may be formed from but is not limited to the following materials: Nickel titanium alloys, for example, nitinol, stainless steel, cobalt alloys and titanium alloys. The loops of the constraining members may be made from common suture material as known in the art, for example polyester suture such as 4-0 Tevdek®, nylon, silk, polypropylene, ultra high molecular weight polyethylene (UHMPE) and the like. The sutures may be monofilament, braided, twisted or multifilament. The loops and the retaining wires may also be made from a metallic alloy such as stainless steel or nickel titanium. In some embodiments, the stent, the loops and/or the retaining wires may be made from biodegradable materials. A number of bioabsorbable homopolymers, copolymers, or blends of bioabsorbable polymers are known in the medical arts. These include, but are not necessarily limited to, polyesters including poly-alpha hydroxy and poly-beta hydroxy polyesters, polycaprolactone, polyglycolic acid, polyether-esters, poly(p-dioxanone), polyoxaesters; polyphosphazenes; polyanhydrides; polycarbonates including polytrimethylene carbonate and poly(iminocarbonate); polyesteramides; polyurethanes; polyisocyanates; polyphosphazines; polyethers including polyglycols, polyorthoesters; epoxy polymers including polyethylene oxide; polysaccharides including cellulose, chitin, dextran, starch, hydroxyethyl starch, polygluconate, hyaluronic acid; polyamides including polyamino acids, polyester-amides, polyglutamic acid, poly-lysine, gelatin, fibrin, fibrinogen, casein, collagen.

Other suitable biocompatible materials may also be used for any of the components described herein.

Operation of the stent delivery systems of the presently-described embodiments is described with reference to the stent delivery system 10 by way of non-limiting example. Alternative methods of operating the system may also be used. The stent delivery system 10 may be provided in a sterile packaging. The stent 28 may be provided in the expanded configuration 66 or constrained configuration 40 within the packaging. For example, some stent materials may weaken or otherwise deform when stored in a constrained configuration 40 with the longitudinal tension exerting force on the stent during shipping and storage. In some embodiments provided with an outer sheath 32, the outer sheath 32 may be provided to hold the stent 28 in position on the stent region 30 without having the proximal and distal constraining members 44, 46 tensioning the stent. For example, the system 10 may be provided with the handle 26 in the open position 64 and the outer sheath 32 over the stent 28 on the inner shaft 22. Prior to insertion of the distal portion 31 of the system 10 into the patient, the operator may move the handle 26 to the closed position 52 and place longitudinal tension on the stent 28 using the proximal and distal constraining members 44, 46 to constrain the stent 28 against the inner shaft 22. The stent 28 may be provided in the expanded configuration 66 in the absence of a sheath as well and be moved to the constrained configuration 40 by operation of the handle 26 to the closed position 52 prior to delivery to the patient.

Minimal fluoroscopy may be used for placement of the stent 28 within the patient lumen because of the simultaneous release of the stent. The simultaneous release of the stent 28 means that the midpoint of the stent 28 in the constrained configuration 40 on the inner shaft 22 is also the midpoint when the stent 28 is released, so that the stent 28 may precisely be positioned based on the known midpoint of the stent 28. Fluoroscopy is not required during placement of the stent 28 once the placement position has been determined. The stricture length within the patient lumen at the treatment site is measured using fluoroscopy. Then the stent 28 may be placed at the proper position within the lumen using an endoscope alone.

The outer sheath 32 may include two different sets of distance measurement markings 37, 39, one to be used when the outer sheath 32 is covering the stent 28 and one set to be used when the outer sheath 32 has been withdrawn and locked to the handle 26 (See FIGS. 2 and 3). The markings 37, 39 may be of different colors, for example, to easily identify the two measurements. The operator measures the distance from the incisor teeth to the midpoint of the stricture. The stent delivery system 10 is inserted into the patient's alimentary canal via the mouth using the first set of sheath markings 37 to place the constrained stent 28 in the stricture by measuring the distance relative to the incisor teeth. The sheath 32 is withdrawn proximally and locked to the handle 26 to expose the stent 28. The second set of markings 39 may be used once the sheath 32 is withdrawn to measure the distance between the stricture and the incisor teeth to ensure that the stent 28 is still in the correct position relative to the stricture. Because the outer sheath 32 is not used to deploy the stent 28, the markings 37, 39 can be placed clearly on the outside of the sheath and the outer sheath can be locked to the handle 26 and held steady relative to the patient's incisor teeth to increase the accuracy of the stent placement.

An endoscope may be positioned within the patient lumen so the operator can view the proximal side of the stricture. The guidewire 36 is inserted through the stricture and the endoscope is removed. The proper length stent 28 is selected based on the stricture measurement. The operator inserts the distal portion 31 of the stent delivery system into the patient lumen with the stent 28 in the constrained configuration 40 on the inner shaft 22. The guidewire 36 may be inserted first to navigate a tortuous pathway to the treatment site and the system 10 is delivered over the guidewire 36 to the treatment site. The endoscope may then be placed into the patient lumen adjacent and parallel to the system 10. Alternatively, the stent delivery system 10 may be inserted into the patient lumen through the working channel of an endoscope, depending on the size and location of the lumen.

A viewing port of the endoscope is used to identify the proximal end of the stricture at the treatment site. The stent region 30 is positioned within the lumen at the treatment point. For embodiments having a softer inner shaft 22, the stiffening member 67 is inserted through the lumen 69 of the inner shaft 22 to provide support for the longitudinally tensioned stent. The outer sheath 32, if present, is proximally withdrawn and the stent 28 in the constrained configuration 40 is exposed within the patient lumen. The constrained stent 28 may be moved within the lumen to correctly position the stent 28 at the implant/treatment site. The stent 28 is moved to the expanded configuration 66 by movement of the handle portion 58 to the open position 64 that moves the proximal and distal constraining members 44, 46 to the second position 49 releasing the longitudinal tension on the stent 28. The position of the expanded stent 28 is monitored using the endoscope. The stent 28 may be returned to the constrained configuration 40 by the operator moving the proximal portion 58 of the handle 26 to the closed position 52 and returning the proximal and distal constraining members 44, 46 to the first position 47 to longitudinally tension the stent 28 against the inner shaft 22, for example if the stent 28 is incorrectly positioned. The stent 28 may be moved from the constrained configuration 40 to the expanded configuration 66 as many times as needed.

Once the proper position for the stent 28 is achieved within the patient lumen, the proximal and distal retaining wires 78, 88 may be proximally withdrawn from the stent 28 to completely release the stent 28 from the proximal and distal constraining members 44, 46. The delivery system 10 is withdrawn proximally from the patient and the endoscope removed.

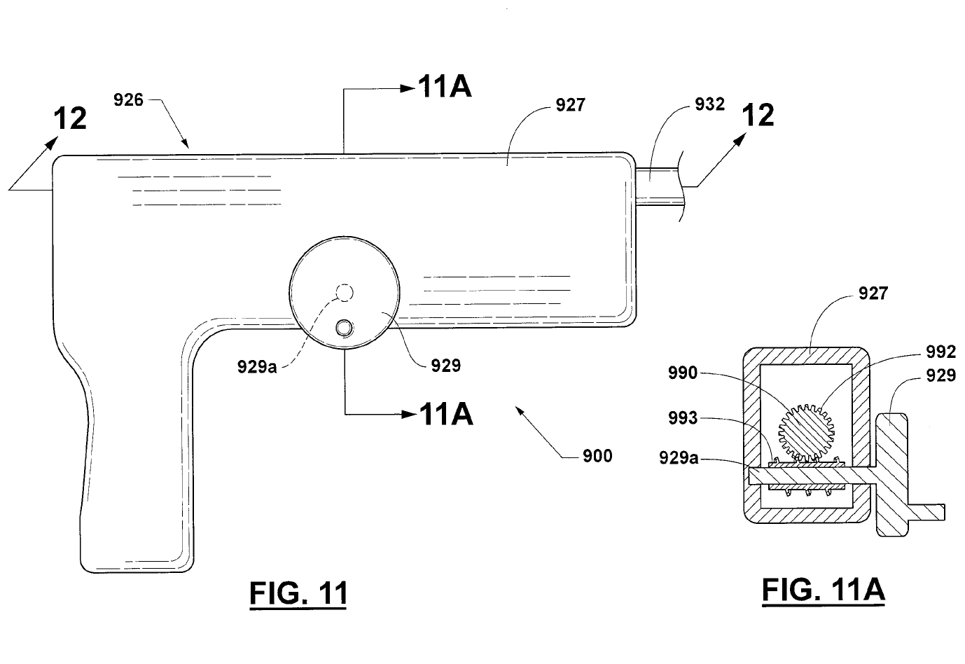

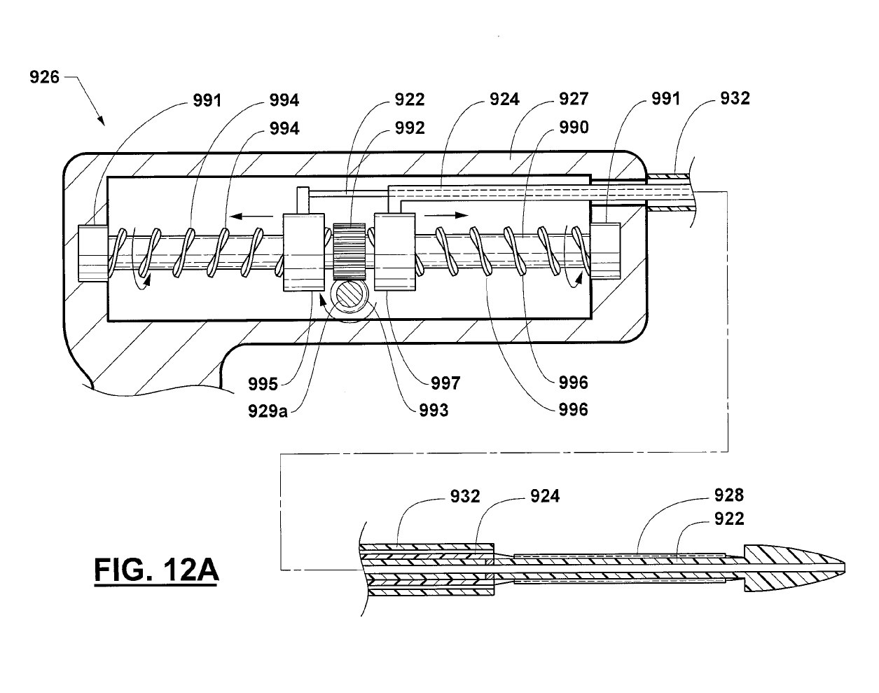

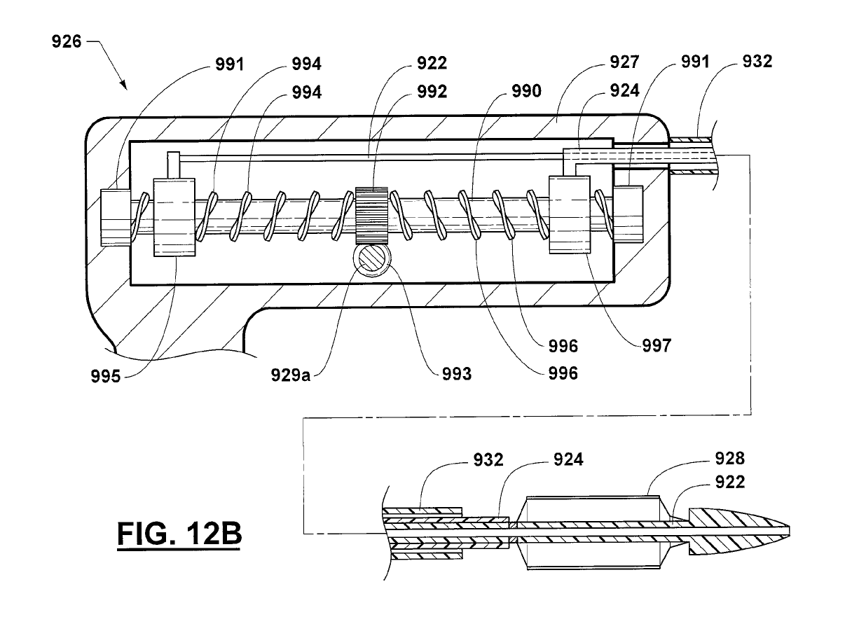

Another stent deployment system 900, including a rotary linear deployment handle mechanism, is illustrated with reference to FIGS. 11-12B. FIG. 11 shows a distally-truncated exterior side view of the system 900 including the enclosed body 927 of the handle 926. The stent delivery system 900 may include an outer sheath 932 slidably positionable over a distal portion of the inner shaft 922 to cover the stent 928. The outer sheath 932, centrally-constrained stent 928, and inner shaft 922 are shown, as is a rotary deployment/recapture handle element 929. FIG. 11A shows a transverse section view of the handle 926 along line 11A-11A, and FIGS. 12A-12B show a longitudinal section view of the main handle body 926 of the device along line 12-12.

The general principle of operation for stent deployment of this embodiment 900 is substantially the same as described with reference to other embodiments above. The mechanisms for stent attachment and release to the inner and outer shafts may be any of those addressed in this and/or other disclosures including U.S. provisional application Ser. No. 61/299,605, filed Jan. 29, 2010, which is incorporated by reference herein in its entirety. In contrast with the lever/trigger-based actuation of inner and/or outer shafts of the other embodiments described herein, the present embodiment 900 provides a rotary handle element 929 configured to operate a linear drive system that actuates the inner and outer shafts 922, 924 to deploy and/or recapture a stent 928 not yet fully released therefrom.

In the embodiment shown here, the rotary handle member 929 includes an axle 929a disposed transverse to the long axis of the handle 926. A worm screw 993 is disposed around a central circumferential portion of the axle 929a and is configured to provide mechanical communication between the rotary handle 929 and the drive shaft 990. It should be appreciated in view of the present disclosure that other embodiments may be practiced within the scope of the present invention (such as, for example, a rotary handle with an axle that is continuous, coaxial, and/or co-linear with the long axis of the drive shaft such that its rotation would be more directly mechanically communicated thereto).

The handle 926 includes an outer housing body 927. A central drive shaft 990 is mounted longitudinally in the body 927 between bearings 991 that are configured to allow bi-directional axial rotation of the drive shaft 990. The drive shaft 990 includes an integrated gear member 992 with teeth configured to interface with a worm screw 993. The drive shaft gear 992 is shown centered along the length of the drive shaft, but it may be located elsewhere along its length. The worm gear 993 is configured to translate rotation of the rotary handle 929 into rotation of the drive shaft 990 about its longitudinal axis. The drive shaft 990 includes proximal surface threads 994 that are oriented opposite distal surface threads 996. A proximal bushing 995 is configured to interface with the proximal threads 994 (e.g., with a complementarily-threaded surface, ball bearing system, or some other cam-following means) such that—when the drive shaft 990 is rotated in a first direction, the proximal bushing 995 is retracted proximally, but advances distally when the shaft 990 is rotated the other way. Likewise, a distal bushing 997 is configured to interface with the distal threads 996 (e.g., with a complementarily-threaded surface, ball bearing system, or some other cam-following means) such that—when the drive shaft 990 is rotated in a first direction—the bushing 995 is advanced distally, but is retracted proximally when the shaft 990 is rotated the other way.

The proximal bushing 995 is attached to the proximal end of the inner shaft 922 (and thereby to the distal end of the stent 928). The distal bushing 997 is attached to the proximal end of the outer shaft 924 (and thereby to the proximal end of the stent 928). Accordingly, when the drive shaft 990 is rotated in the first direction by operation of the rotary handle 929, the proximal bushing 995 retracts the inner shaft 922 proximally, while—at the same time—the distal bushing 997 advances the proximal end of the stent 928 distally. The opposite is true when the drive shaft 990 is rotated in the second direction. These functions are described below with reference to FIGS. 12A and 12B.

FIG. 12A shows the device 900 with the stent 928 in a constrained configuration. As noted above, the distal end of the stent 928 is attached to the elongate inner shaft 922, which is longitudinally slidable relative to the handle 910. The proximal end of the stent 928 is attached to the elongate tubular outer shaft 924, which is disposed coaxially around and longitudinally slidable relative to the inner shaft 922. As described above with reference to FIGS. 11-12B, the rotary gear mechanism elements are also disposed within the handle housing 927. When the stent 928 is in the constrained configuration of FIG. 12A, the bushings 995, 997 are nearer the central gear 993.

To actuate the device 900 and deploy/expand the stent 928, a user may turn the rotary handle 929 to rotate the worm gear 995. The mechanical communication of the worm gear 995 with the central gear 993 rotates the drive shaft 990 about its longitudinal axis. As described above, the rotary action of the distal threads 996 in mechanical communication with the distal bushing 997 moves the distal bushing 997 distally, and the rotary action of the proximal threads 994 in mechanical communication with the proximal bushing 995 simultaneously moves the proximal bushing 995 proximally. Motion arrows in FIG. 12A are used to indicate the motion of these components and reach the configuration shown in FIG. 12B. The threads 994, 996 are shown as being symmetrical, but it should be appreciated that—in other embodiments—the proximal and distal threads may be sized/pitched differently to provide a corresponding differential motion between the proximal and distal stent ends.

FIG. 12B shows the stent 928 in an expanded configuration—that is partially deployed, but not released from its releasable attachments to the inner and outer shafts. The proximal and distal bushings 995, 997 are now further apart from each other and nearer the proximal and distal ends, respectively, of the handle. The mechanism confers mechanical advantage by simultaneously imparting proximal and dual movement to deploy/release or recapture a stent with less linear handle movement than the same deployment/recapture operation would require with a lever mechanism (e.g., such as those described above). This design may therefore be particularly useful for stents that are very long and/or that have high foreshortening percentages, with modification to the length and/or threading of the drive shaft). In view of the present disclosure, it will be appreciated that the size and pitch of the threads on the worm gear, central gear and/or the drive shaft, as well as the size of the drive shaft may be modified within the skill in the art to provide different levels of mechanical advantage and efficiency.

If the stent 928 needs recaptured and/or otherwise reduced in outer diameter to be repositioned, the process may be reversed, turning the rotary handle 929 in the opposite direction to move the bushings 995, 997 and the stent 928 back to the position shown in FIG. 12A, or at least a position with a smaller outer stent diameter than is depicted in FIG. 12B. Visual and/or tactile indicia may be provided on the device to provide information to a user regarding the distance that the inner and/or outer shafts are moving.

The above figures and disclosure are intended to be illustrative but not exhaustive. This description may suggest many variations and alternatives to one of ordinary skill in the art, which may be practiced within the scope of the attached claims. Those familiar with the art may recognize other equivalents to the specific embodiments described herein which equivalents are intended to be encompassed by the attached claims, as are the various possible combinations of different elements of embodiments described in the present application.

We claim:

1. A stent delivery system comprising:
an elongate outer tubular shaft including a proximal portion, a distal portion, and a stent attachment portion on the distal portion of the outer shaft, said stent attachment portion configured for attachment to a proximal stent end;
an elongate inner shaft extending longitudinally, coaxially, slidably through the outer elongate tubular shaft including a proximal portion, a distal portion, and a stent receiving portion on the distal portion of the shaft, said stent receiving portion configured for attachment to a distal stent end;
a housing configured to house a stent-deployment mechanism; and
a stent deployment mechanism disposed in the housing, the mechanism comprising:
a rotatable handle configured to be rotatable relative to the housing;
a drive member having a first threaded portion and a second threaded portion, the drive member being disposed in mechanical communication with the rotatable handle and configured to be rotated about its longitudinal axis by said mechanical communication upon an actuation of the rotatable handle;
a first drive-engaging member attached to a proximal portion of the inner shaft and comprising a first drive-engaging surface configured to engage the first threaded portion of the drive member; and
a second drive-engaging member attached to a proximal portion of the outer shaft, and comprising a second drive-engaging surface configured to engage the second threaded portion of the drive member; and
wherein the inner shaft and the outer shaft are configured to cooperatively apply opposing longitudinal tensile forces to at least a portion of a stent in the constrained configuration with the proximal and distal constraining members each in a first position corresponding to a first relative position of the movable handle, first drive-engaging member, and second drive-engaging member.

2. The stent delivery system of claim 1, wherein the mechanical communication between the rotatable handle and the drive member comprises a gear member.

3. The stent delivery system of claim 1, wherein the mechanical communication between the rotatable handle and the drive member comprises a worm gear configured to engage a toothed gear surface integrated with the drive member.

4. The stent delivery device of claim 1, wherein the first and second threaded portions of the drive member and the first and second drive-engaging members are respectively configured such that rotation of the drive member about its longitudinal axis in a first direction will move the drive-engaging members closer together.

5. The stent delivery system of claim 1, wherein the first and second drive-engaging members are each configured as bushings including a cam-following structure configured to follow, respectively, the first and second drive member threaded portions.

6. The stent delivery system of claim 1, further comprising a stent attached to the inner and outer shafts, the stent having a constrained configuration and an expanded configuration.

7. The stent delivery system of claim 6, wherein the stent is repeatedly movable between the constrained configuration and the expanded configuration.

8. The stent delivery system of claim 6, wherein the stent is configured as an esophageal stent.

9. The stent delivery system of claim 6, further comprising a sheath removably positionable over the stent and a portion of the elongate shaft.

10. The stent delivery system of claim 6, further comprising a distal sheath positionable over a distal portion of the stent.

11. The stent delivery system of claim 6, wherein the outer shaft and the inner shaft are configured to mechanically communicate through the first and second racks and across the cog to move in opposite directions in relation to each other, thereby to move the proximal and distal stent ends in opposite directions in relation to each other to expand or constrain the stent depending upon the movement direction.

12. The stent delivery system of claim 1, further comprising a sheath removably positionable over the stent and a portion of the elongate shaft.

13. The stent delivery system of claim 1, further comprising a distal sheath positionable over a distal portion of the stent.

14. A handle for a stent delivery system, the handle comprising:
a housing configured to house components of a stent deployment mechanism;
a rotatable handle member rotatably attached to the housing;
a drive shaft disposed in mechanical communication with the rotatable handle, the drive shaft disposed longitudinally in the housing and configured to be rotatable about a longitudinal axis of the drive shaft;
a first shaft attached near a distal first shaft end to a stent, and attached near a proximal first shaft end to a first bushing, which first bushing is configured in mechanical communication with the drive shaft so as to be longitudinally movable in a first direction thereby upon a rotation of the drive shaft; and
a second shaft attached near a distal second shaft end to the stent, and attached near a proximal second shaft end to a second bushing, which second bushing is configured in mechanical communication with the drive shaft so as to be longitudinally movable thereby in a second direction—opposite the first direction—upon the rotation of the drive shaft.

15. The handle of claim 14, wherein the stent is configured as an esophageal stent.

16. A method of implanting a stent in a patient lumen, the method comprising:
providing a stent delivery system including a handle according to claim 14;
inserting a distal portion of a stent delivery system into a lumen of a patient, the stent delivery system configured wherein
the first shaft is configured as an inner shaft, a distal portion of which is attached to a distal portion of the stent, and
the second shaft is configured as an outer shaft disposed slidably coaxially around a lengthwise portion of the inner shaft, a distal portion of the outer shaft being attached to a proximal portion of the stent, and
the stent configured with a constrained configuration and an expanded configuration;
holding the stent in the constrained configuration with longitudinal tensile force applied to the stent by the first and second shafts, each located in a first position;
directing the stent to an implant site;
deploying the stent to the expanded configuration by moving the first and second rack shafts to a second position by actuation of the rotatable handle member in a manner moving the inner shaft proximally, the outer shaft distally, and thereby releasing longitudinal force on the stent.

17. The method of claim 16, further comprising reapplying longitudinal force to the stent to move the stent from the expanded configuration to the constrained configuration by rotating the rotatable handle in an opposite direction and thereby moving the inner and outer shafts toward the first position.

18. The method of claim 16, wherein the stent is configured as an esophageal stent.

19. The method of claim 16, further comprising providing a removable sheath over the stent and a portion of the inner shaft and withdrawing the sheath from the stent in the patient lumen such that the stent is exposed.

20. The method of claim 16, wherein the mechanical communication between the rotatable handle and the drive shaft comprises a worm gear.
